(19) United States
(12) Patent Application Publication
(10) Pub. No.: US 2025/0259268 A1
(43) Pub. Date: Aug. 14, 2025

(54) IMAGE PROCESSING METHOD AND INFORMATION PROCESSING APPARATUS

(71) Applicant: Fujitsu Limited, Kawasaki-shi (JP)

(72) Inventors: Fuya NAKAMURA, Ota (JP); Masaki TAKEUCHI, Kawasaki (JP)

(73) Assignee: Fujitsu Limited, Kawasaki-shi (JP)

(21) Appl. No.: 19/190,938

(22) Filed: Apr. 28, 2025

Related U.S. Application Data
(63) Continuation of application No. PCT/JP2022/041108, filed on Nov. 4, 2022.

(51) Int. Cl. G06T 5/40; G06T 5/60; G06T 7/00

(52) U.S. Cl. CPC G06T 5/40; G06T 5/60; G06T 7/0016; G06T 2207/10081; G06T 2207/20081; G06T 2207/20084; G06T 2207/30016; G06T 2207/30096

(57) ABSTRACT

An information processing apparatus generates a histogram indicating the number of pixels for an individual luminance value of a first image obtained by capturing an image of a first subject under a first imaging condition, generates a histogram indicating the number of pixels for an individual luminance value of a second image obtained by capturing an image of a second subject of a same type as the first subject under a second imaging condition, generates a conversion rule for the luminance values of the pixels of the second image to improve a similarity between the histograms of the first image and the second image, converts a luminance value of an individual pixel of a third image obtained by capturing an image of a third subject of the same type under the second imaging condition, by using the conversion rule, and executes machine learning by using the third image.

### CROSS-REFERENCE TO RELATED APPLICATION

This application is a continuation application of International Application PCT/JP2022/041108 filed on Nov. 4, 2022, which designated the U.S., the entire contents of which are incorporated herein by reference.

### FIELD

The present embodiments relate to an image processing method and an information processing apparatus.

### BACKGROUND

There is an image analysis technique using artificial intelligence (AI). For example, in the case of a medical institution, AI is able to diagnose the presence or absence of a lesion, based on a computed tomography (CT) image. In AI-based image analysis, a large number of training images need to be collected in order to generate a model (for example, a neural network) for image analysis. If the similarity among the imaging conditions of the collected images is higher, more accurate learning is performed. However, in some cases, it is difficult to prepare images that have been captured under similar imaging conditions for learning. For example, because different CT imaging apparatuses have different versions and settings, these apparatuses also have different image reconstruction parameters. Therefore, there is a subtle difference in how an image appears for each body tissue. This difference in appearance causes deterioration in accuracy of a machine learning model.

By performing common preprocessing on a plurality of images, it is possible to reduce the difference in how the images appear. As an image conversion technique, for example, there has been proposed an image processing apparatus capable of improving correction accuracy to obtain an image close to human vision (human eye).

See, for example, Japanese Laid-open Patent Publication No. 2021-152686.

### SUMMARY

In one aspect, there is provided a non-transitory computer-readable recording medium storing therein a computer program that causes a computer to execute a process including: generating a histogram indicating a number of pixels for an individual luminance value of a first image obtained by capturing an image of a first subject under a first imaging condition; generating a histogram indicating a number of pixels for an individual luminance value of a second image obtained by capturing an image of a second subject of a same type as the first subject under a second imaging condition; generating a conversion rule for the luminance values of the pixels of the second image, the conversion rule improving a similarity between the histogram of the first image and the histogram of the second image; converting a luminance value of an individual pixel of a third image obtained by capturing an image of a third subject of the same type as the first subject under the second imaging condition, by using the conversion rule; and executing machine learning by using the third image whose luminance values have been converted.

The object and advantages of the invention will be realized and attained by means of the elements and combinations particularly pointed out in the claims.

It is to be understood that both the foregoing general description and the following detailed description are exemplary and explanatory and are not restrictive of the invention.

### BRIEF DESCRIPTION OF DRAWINGS

FIG. 1 is a diagram illustrating an example of an image processing method according to a first embodiment;

FIG. 2 is a diagram illustrating an example of a system configuration according to a second embodiment;

FIG. 3 is a diagram illustrating an example of hardware of a server;

FIG. 4 is a diagram illustrating an example of how an image captured by an individual examination apparatus appears;

FIG. 5 is a diagram illustrating an example of preprocessing to be uniformly applied to an entire image;

FIG. 6 is a diagram illustrating an example of preprocessing that improves the similarity between histograms of luminance values;

FIG. 7 is a block diagram illustrating functions of an individual apparatus for image diagnosis support;

FIG. 8 is a diagram illustrating an example of an image diagnosis support method using images captured under different imaging conditions;

FIG. 9 is a diagram illustrating an example of a procedure for generating a conversion formula;

FIG. 10 is a flowchart illustrating an example of a processing procedure in a preprocessing phase;

FIG. 11 is a flowchart illustrating an example of a processing procedure of a learning phase;

FIG. 12 is a diagram illustrating an example of image diagnosis information distribution processing;

FIG. 13 is a diagram illustrating an example of inference processing; and

FIG. 14 is a flowchart illustrating an example of a processing procedure of an inference phase.

### DESCRIPTION OF EMBODIMENTS

Even if a plurality of images captured under different imaging conditions are converted by using conventional preprocessing, there are cases in which a processing result suitable for use in machine learning is not obtained. Therefore, when machine learning needs to be performed by using images captured under different imaging conditions, it is difficult to perform accurate machine learning due to the difference in appearance of the images.

Hereinafter, embodiments will be described with reference to the drawings. Each embodiment may be implemented by combining a plurality of embodiments within a consistent range.

### First Embodiment

The first embodiment is an image processing method for reducing the difference in appearance among images used for machine learning.

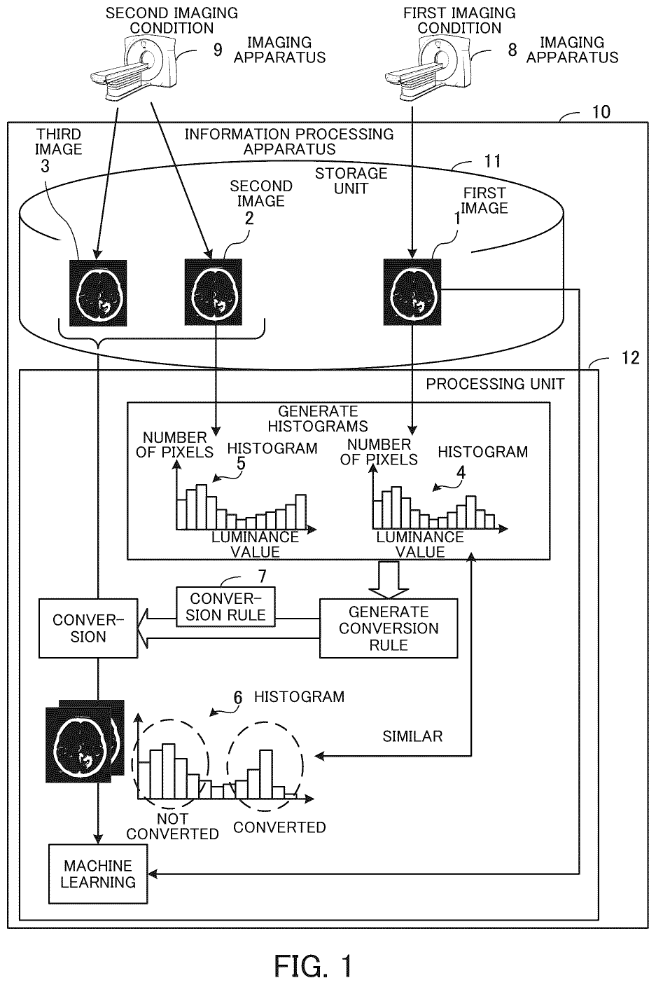

FIG. 1 is a diagram illustrating an example of an image processing method according to the first embodiment. FIG. 1 illustrates an information processing apparatus 10 that implements an image processing method. The information processing apparatus 10 may implement an image processing method according to the first embodiment by executing, for example, an image processing program.

The information processing apparatus 10 processes images captured by imaging apparatuses 8 and 9. The individual image captured is represented by image data including a luminance value of an individual pixel. The information processing apparatus 10 includes a storage unit 11 and a processing unit 12. The storage unit 11 is, for example, a memory or a storage device included in the information processing apparatus 10. The processing unit 12 is, for example, a processor or an arithmetic circuit included in the information processing apparatus 10.

The information processing apparatus 10 converts the luminance values such that each of the plurality of images used for machine learning has a similar shape of the distribution (histogram) of the number of pixels for each luminance value. In this way, the difference in appearance among the images is reduced. That is, in the machine learning, higher accuracy is obtained if the similarity among the histograms among the luminance values of the pixels of a plurality of images is higher. For example, in machine learning for detecting the part of a lesion from a captured CT image displaying a cross section of the brain of a human body, if the similarity among the histograms is higher, more accurate learning is performed. Conversely, when there is a difference among the histograms of the plurality of images used for machine learning, the accuracy of inference by a trained machine learning model may decrease due to the difference among the histograms. In this case, it is conceivable to correct the luminance of each image to make the distributions of the number of pixels for each luminance value similar to each other.

However, in the preprocessing of the related art, since uniform processing is performed on an entire image, there are cases in which the image is not converted into an image suitable for learning by machine learning. For example, there may be a case where, among a plurality of images, there is no large difference in the number of pixels for each luminance in a low luminance range, but there is a large difference in the number of pixels for each luminance in a high luminance range. In this case, even if uniform luminance correction is performed on the entire image in order to uniformize the distributions of the number of pixels for each luminance, it is difficult to improve the similarity of the luminance distribution among the images.

For example, in the case of CT imaging, an imaging target subject is a part of a human body. In a case where there are a plurality of captured images of the same type of subject, how the images appear differs depending on the difference in the subject itself (presence or absence of a lesion or the like) and the imaging conditions at the time of imaging. In a case where machine learning is performed with such a difference in the subject itself, it is important to sufficiently reduce the difference in the appearance due to the imaging conditions. Therefore, the information processing apparatus 10 generates a conversion rule 7 for making the histograms of the luminance values among the images similar to each other in association with the imaging conditions, and reduces the difference in appearance among the images by image conversion using the conversion rule 7. The details are as follows.

The storage unit 11 stores, for example, images acquired from the two imaging apparatuses 8 and 9. For example, the storage unit 11 stores a first image 1, a second image 2, and a third image 3. The imaging apparatus 8 has captured an image of a first subject under a first imaging condition, and this image is used as the first image 1. The imaging apparatus 9 has captured an image of a second subject of the same type as the first subject under a second imaging condition, and this image is used as the second image 2. The imaging apparatus 9 has captured an image of a third subject of the same type as the first subject under the second imaging condition, and this image is used as the third image 3.

The processing unit 12 generates a histogram 4 indicating the number of pixels for an individual luminance value of the first image 1 obtained by capturing an image of the first subject under the first imaging condition. In addition, the processing unit 12 generates a histogram 5 indicating the number of pixels for an individual luminance value of the second image 2 obtained by capturing an image of the second subject of the same type as the first subject under the second imaging condition.

Next, the processing unit 12 generates the conversion rule 7 for the luminance values of the pixels of the second image 2. The conversion rule 7 is a luminance value conversion rule that improves the similarity between the histogram 4 of the first image 1 and the histogram 5 of the second image 2. By converting the luminance value of each pixel of the second image 2 with the conversion rule 7, the shape of the histogram of the image pixel luminance values converted becomes similar to the histogram 4 of the first image 1.

The conversion rule 7 generated based on the second image 2 may also be applied to the third image 3 captured under the same second imaging condition as those of the second image 2. Therefore, the processing unit 12 converts the luminance value of an individual pixel of the third image 3 obtained by capturing an image of the third subject of the same type as the first subject under the second imaging condition by using the conversion rule 7. The shape of the histogram 6 of the luminance values of the pixels of the third image 3 converted is similar to that of the histogram 4 of the first image 1.

Then, the processing unit 12 executes machine learning by using the first image 1, the second image 2, and the third image 3. For example, it is assumed that a plurality of first images 1 and a plurality of second images 2 are prepared, and correct labels for training are added to the first images 1 and the second images 2. In this case, the processing unit 12 executes the learning phase of the supervised machine learning, based on the images obtained by converting the luminance value of each pixel of the second images 2 with the conversion rule 7 and the first images 1. As a result, a trained machine learning model (learned model) is generated. The processing unit 12 executes the inference phase of the machine learning by using the third image 3 whose luminance values have been converted, and by using the generated learned model.

By converting the luminance value of each pixel of the individual second image 2 or third image 3 captured under the second imaging condition with the conversion rule 7 in this manner, the difference in how the first images 1, the second images 2, and the third image 3 appear is reduced. That is, it is possible to prepare a large number of images with little difference in appearance. As a result, for example, the second images 2 or the third image 3 is effectively used for machine learning using the first images 1, and the accuracy of machine learning is improved.

The processing unit 12 may generate the conversion rule 7 in which the range of the luminance values to be converted is limited. In this case, in the process of converting the luminance value of each pixel of the third image 3, the processing unit 12 converts the luminance value of an individual pixel within the range limited in the conversion rule 7 in accordance with the conversion rule 7. As a result, it is possible to generate the conversion rule 7 in which, for example, only a range of luminance values having a difference in shape between the histograms 4 and 5 is a conversion target. By limiting the range of the luminance values to be converted, the conversion rule 7 for making the shapes of the histograms 4 and 5 similar within the range is easily generated, and the similarity between the histograms 4 and 5 is further increased. That is, it is possible to further reduce the difference in appearance, and it is possible to improve the accuracy of machine learning.

Further, the processing unit 12 is also able to optimize constants by repeatedly executing a process of correcting the constants included in the conversion formula of the luminance values of the pixels of the second image 2 such that the similarity between the shapes of the histogram 4 of the first image 1 and the histogram 5 of the second image 2 is improved. In this case, the processing unit 12 sets the conversion formula in which the constants have been optimized as the conversion rule 7. By generating the conversion rule 7 through the optimization of the constants of the conversion formula in this manner, it becomes easy to generate the conversion rule 7 more accurately, and the similarity between the histograms is further increased.

The processing unit 12 may use, as the constants to be optimized, for example, a first constant included in a conversion formula for calculating a post-conversion luminance value from a pre-conversion luminance value and a second constant indicating a range of luminance values to be converted. In this case, the processing unit 12 converts the luminance values of the pixels in the range indicated by the second constant, based on the conversion formula. This makes it possible to accurately calculate a range of luminance values appropriate as a conversion target, and to further increase the similarity between histograms.

The processing unit 12 may refer to the number of images captured under their respective imaging conditions, determine one of the plurality of imaging conditions as the first imaging condition based on the number of images, and determine an image captured under the first image capture condition as the first image 1. For example, an imaging condition under which more images are prepared for machine learning is determined as the first imaging condition. As the number of images captured under the same imaging condition increases, machine learning for the images captured under the imaging condition is performed more accurately. The accuracy of machine learning is improved by changing the luminance value of each pixel of images captured under other imaging conditions in accordance with images suitable for highly accurate machine learning.

As machine learning, the processing unit 12 may train a machine learning model that detects a certain part from an input image, by using, for example, the first image 1, the second image 2 whose luminance values have been converted, or the third image 3 whose luminance values have been converted. The training result is output as a learned model. As a result, even if the number of images captured under individual imaging conditions is small, if there are a large number of images captured under any of a plurality of imaging conditions, it is possible to generate a highly accurate learned model.

For example, the processing unit 12 converts the luminance value of an individual pixel of a fourth image obtained by capturing an image of a subject of the same type as the first image 1 under the second imaging condition with the conversion rule 7, and detects the certain part from the fourth image whose luminance values have been converted, by using the learned model. This makes it possible to detect a certain part more accurately by using a common learned model for images captured under different imaging conditions.

It is possible to restate the detection method and the detection program of the certain part by using the conversion rule 7 by the information processing apparatus 10 as follows when the image used for training is the fourth image and the image used for detection of the certain part is the third image 3.

The information processing apparatus 10 detects the certain part included in the third subject by inputting the first converted image obtained by converting the luminance value of each pixel of the third image 3 by using the conversion rule 7 to the trained machine learning model that detects the certain part from the input image. At this time, the conversion rule 7 is a conversion rule for the luminance values of the pixels of the second image 2, the conversion rule improving similarity between the histogram 4 of the first image 1 obtained by capturing an image of the first subject under the first imaging condition and the histogram 5 of the second image 2 obtained by capturing an image of the second subject of the same type as the first subject under the second imaging condition. The third image 3 is an image obtained by capturing an image of a third subject of the same type as the first subject under the second imaging condition. The trained machine learning model (learned model) is a machine learning model trained by machine learning using a converted image obtained by converting a luminance value for each pixel of a fourth image obtained by capturing an image of a fourth subject of the same type as the first subject under the second imaging condition based on the conversion rule 7.

### Second Embodiment

In recent years, a technique of image diagnosis support using AI has attracted attention. It is expected that the image diagnosis support prevents oversight of a lesion and reduces the time needed for a doctor to interpret an image, for example. Therefore, as a second embodiment, a technique for improving diagnosis accuracy in image diagnosis support using AI will be described.

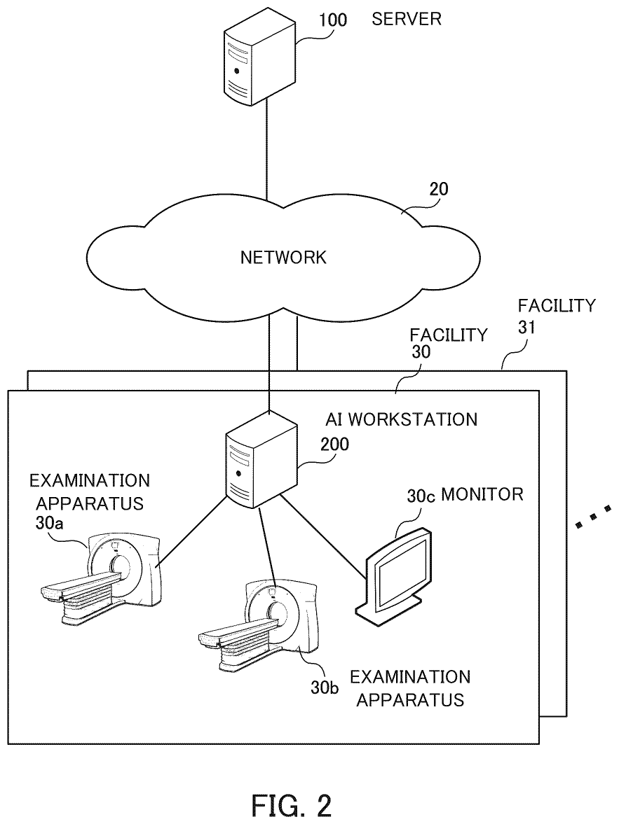

FIG. 2 is a diagram illustrating an example of a system configuration according to the second embodiment. For example, systems in a plurality of facilities 30, 31, etc., are connected to a server 100 via a network 20. The server 100 is a computer that performs learning by AI.

In the facility 30, a system including an AI workstation 200, examination apparatuses 30a and 30b, and a monitor 30c is constructed. The AI workstation 200 is connected to the network 20. The AI workstation 200 is a computer that performs diagnosis support based on medical images by using a learned model.

The examination apparatuses 30a and 30b are connected to the AI workstation 200. The examination apparatuses 30a and 30b are used for examination of patients, and output images as examination results. The examination apparatuses 30a and 30b are, for example, CT apparatuses, magnetic resonance imaging (MRI) apparatuses, or the like. The examination apparatuses 30a and 30b are examples of the imaging apparatuses 8 and 9 according to the first embodiment.

The monitor 30c is connected to the AI workstation 200. The monitor 30c acquires, for example, images captured by the examination apparatuses 30a and 30b and images indicating analysis results from the AI workstation 200 and displays these images.

A system similar to that of the facility 30 is also constructed in each of the facilities 31, etc., other than the facility 30. This system including the server 100 and the AI workstations of the facilities 30, 31, etc., is an example of the information processing apparatus 10 according to the first embodiment.

The server: that has collected annotated images from the facilities 30, 31, etc., performs learning using the collected images and generates a learned model. The generated model is transmitted from the server 100 to the facilities 30, 31, etc., and is used in the systems in the facilities 30, 31, etc. For example, the AI workstation 200 of the facility 30 analyzes the images acquired from the examination apparatuses 30a and 30b by using the model acquired from the server 100, and detects the presence or absence of a lesion.

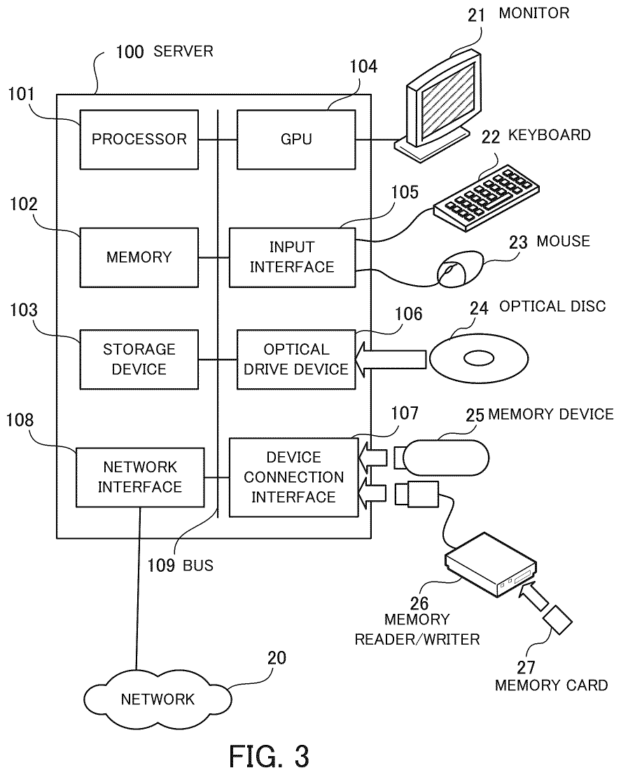

FIG. 3 is a diagram illustrating an example of hardware of the server. The entire server 100 is controlled by a processor 101. A memory 102 and a plurality of peripheral devices are connected to the processor 101 via a bus 109.

The server 100 may be a multiprocessor system including a plurality of processors. A set of processors in a multiprocessor system may be referred to as a processor 101. The processor 101 may be referred to as processor circuitry. Each of the plurality of processors may execute some or all of the plurality of processes executed by the server 100. When there are a plurality of related processes, two or more processes among the plurality of processes may be executed by different processors.

The processor 101 is, for example, a central processing unit (CPU), a micro processing unit (MPU), or a digital signal processor (DSP). At least a part of the functions implemented by the processor 101 executing a program may be implemented by an electronic circuit such as an application specific integrated circuit (ASIC) or a programmable logic device (PLD).

The memory 102 is used as a main storage device of the server 100. The memory 102 temporarily stores at least part of an operating system (OS) program and application programs to be executed by the processor 101. The memory 102 also stores various data used for processing by the processor 101. As the memory 102, for example, a volatile semiconductor storage device such as a random access memory (RAM) is used.

Examples of the peripheral devices connected to the bus 109 include a storage device 103, a graphics processing unit (GPU) 104, an input interface 105, an optical drive device 106, a device connection interface 107, and a network interface 108.

The storage device 103 electrically or magnetically writes and reads data to and from a built-in recording medium. The storage device 103 is used as an auxiliary storage device of the server 100. The storage device 103 stores OS programs, application programs, and various data. As the storage device 103, for example, a hard disk drive (HDD) or a solid state drive (SSD) may be used.

The GPU 104 is an arithmetic device that performs image processing, and is also called a graphic controller. A monitor 21 is connected to the GPU 104. The GPU 104 displays an image on the screen of the monitor 21 in accordance with an instruction from the processor 101. Examples of the monitor 21 include a display device using organic electro luminescence (EL) and a liquid crystal display device.

A keyboard 22 and a mouse 23 are connected to the input interface 105. The input interface 105 transmits signals sent from the keyboard 22 and the mouse 23 to the processor 101. The mouse 23 is an example of a pointing device, and other pointing devices may be used alternatively. Examples of other pointing devices include a touch panel, a tablet, a touch pad, and a track ball.

The optical drive device 106 reads data recorded on an optical disc 24 or writes data to the optical disc 24 using laser light or the like. The optical disk 24 is a portable recording medium on which data is recorded so as to be readable by reflection of light. The optical disk 24 may be a digital versatile disc (DVD), a DVD-RAM, a compact disc read-only memory (CD-ROM), a CD-Recordable (R)/ReWritable (RW), or the like.

The device connection interface 107 is a communication interface for connecting peripheral devices to the server 100. For example, a memory device 25 and a memory reader/writer 26 may be connected to the device connection interface 107. The memory device 25 is a recording medium having a function of communicating with the device connection interface 107. The memory reader/writer 26 is a device that writes data to a memory card 27 or reads data from the memory card 27. The memory card 27 is a card-type recording medium.

The network interface 108 is connected to the network 20. The network interface 108 transmits and receives data to and from other computers or communication devices via the network 20. The network interface 108 is a wired communication interface connected to a wired communication device such as a switch or a router via a cable. Further, the network interface 108 may be a wireless communication interface that is connected to and communicates with a wireless communication device such as a base station or an access point by radio waves.

The server 100 is able to implement the processing functions according to the second embodiment with the hardware as described above. The AI workstation 200 may also be implemented by hardware similar to that of the server 100. The information processing apparatus 10 according to the first embodiment may also be implemented by hardware similar to that of the server 100.

The server 100 implements the processing functions according to the second embodiment by executing a program recorded in a computer-readable recording medium, for example. The program describing the processing contents to be executed by the server 100 may be recorded in various recording media. For example, a program to be executed by the server 100 may be stored in the storage device 103. The processor 101 loads at least a part of the program in the storage device 103 into the memory 102 and executes the program. The program to be executed by the server 100 may be recorded in a portable recording medium such as the optical disk 24, the memory device 25, or the memory card 27. The program stored in the portable recording medium becomes executable after being installed in the storage device 103 under the control of the processor 101, for example. Alternatively, the processor 101 may read the program directly from the portable recording medium and execute the program.

In order to socially implement image diagnosis support using AI, it is important that deterioration in determination accuracy does not occur in images captured by any examination apparatuses. Hereinafter, the difficulty in suppressing the accuracy deterioration will be described.

For example, in the case of a CT image, characteristics of the image change due to the dose at the time of the image capturing or the filtering process at the time of the image reconstruction. The dose is the amount of X-rays applied to the patient. Although a higher dose produces a sharper image, the higher dose results in a greater exposure of the patient. Therefore, the dose is set for each CT apparatus so that the balance between the sharpness of the image and the exposure dose will be appropriate. The filtering process is a noise removal technique used when an image is formed from X-ray map information. The filtering process method differs depending on the CT apparatus manufacturer, and different methods result in different image appearances.

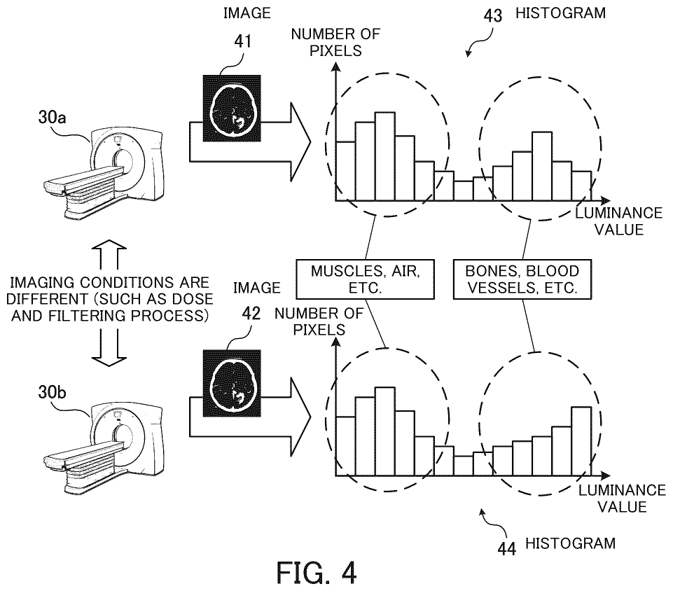

FIG. 4 is a diagram illustrating an example of how an image captured by an individual examination apparatus appears. For example, assuming that the examination apparatus 30a and the examination apparatus 30b have different imaging conditions such as about the dose and filtering process. An image 41 captured by the examination apparatus 30a and an image 42 captured by the examination apparatus 30b have different distributions of the number of pixels for each luminance value.

In the images 41 and 42, a region in which a muscle, air, or the like appears has a low pixel value (the region is dark). On the other hand, in the images 41 and 42, a region in which a blood vessel, a bone, or the like appears has a high luminance value (the region is bright).

For example, since the transmittance of X-rays is different for each body tissue, the luminance of bones, blood vessels, and the like having high transmittance is high, but the luminance of muscles and the like having low transmittance does not change greatly even if there is a difference in dose. The filtering process affects only body tissues (bones, blood vessels, etc.) having high luminance values. Therefore, when histograms 43 and 44 of the luminance values of the images 41 and 42 are compared, there is no large difference in the distribution of the number of pixels for each luminance value in a range of low luminance values.

On the other hand, in a range of high luminance values, a difference in imaging conditions appears between the histograms 43 and 44, and there is a large difference in the distribution of the number of pixels for each luminance value between the histograms 43 and 44. For example, the luminance value corresponding to the largest number of pixels in the range of high luminance values is greatly different between the histogram 43 and the histogram 44.

In such a case, for example, if a sufficient number of images captured by an individual examination apparatus are annotated, it is possible to generate a model optimized for the examination apparatus by using the images of this examination apparatus. However, a skilled doctor takes a sufficient time to perform annotation, and it is not easy to perform annotation on a large number of images. Therefore, it is difficult to secure a sufficient number of annotated images for each examination apparatus.

When there are a plurality of examination apparatuses, if there is a bias in the number of annotated images obtained from the examination apparatuses, the generated model is optimized for an examination apparatus having a large amount of data. As a result, the accuracy of image diagnosis for images captured by an examination apparatus in which annotated images have not sufficiently been prepared decreases.

Therefore, it is conceivable to absorb the difference in how images appear due to a difference in examination apparatus by performing uniform preprocessing when images are learned by AI. Examples of conventional preprocessing include Min-Max normalization, Mean Std normalization, and pixel value filtering. In general, in a CT image, a dose and filtering process have different degrees of influence for each body tissue, and thus uniform preprocessing performed in a conventional way is less effective.

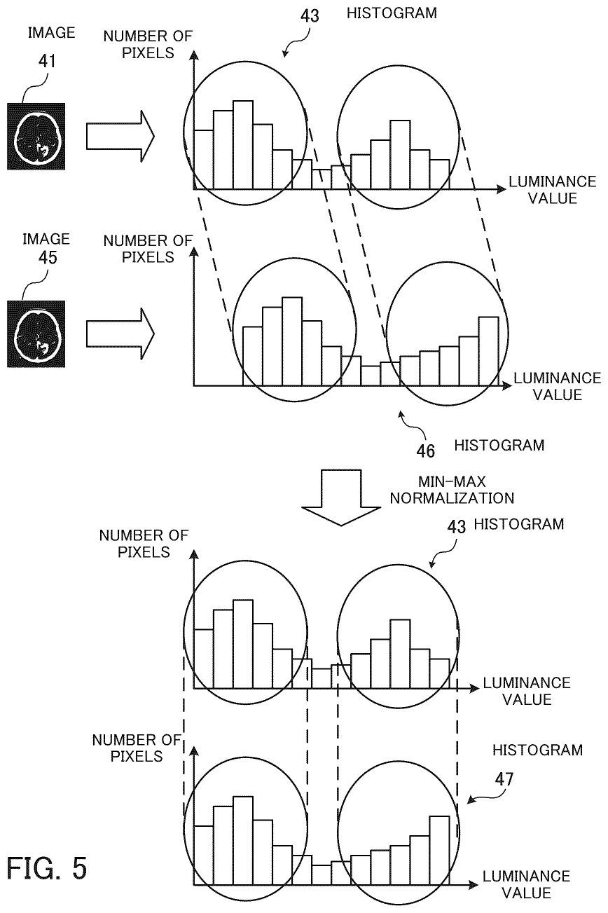

FIG. 5 is a diagram illustrating an example of preprocessing to be uniformly applied to an entire image. In the example of FIG. 5, Min-Max normalization is performed as preprocessing on an image 45. Before the preprocessing is performed, a histogram 46 of the luminance values of the image 45 is biased to a higher luminance side, compared with the histogram 43 of the luminance values of the image 41. When Min-Max normalization is performed on the image 45, the luminance values uniformly decrease as illustrated in a histogram 47. As a result, the range of the luminance values of the histogram 43 of the image 41 matches the range of the luminance values of the histogram 47 normalized. However, the distribution in the range of high luminance values differs between the histogram 43 and the histogram 47.

As described above, in the conventional preprocessing, since all the luminance values change by the same value, the effect of increasing the similarity of the histograms of luminance values is not obtained unless images whose distributions of the histograms are originally similar to each other are used.

Therefore, the server 100 implements preprocessing for maximizing the similarity among the distributions of the histograms of all pixels included in each of the plurality of images used for AI learning or inference. By performing learning and inference using the images to which the preprocessing has been applied, it is possible to improve the accuracy of AI image diagnosis.

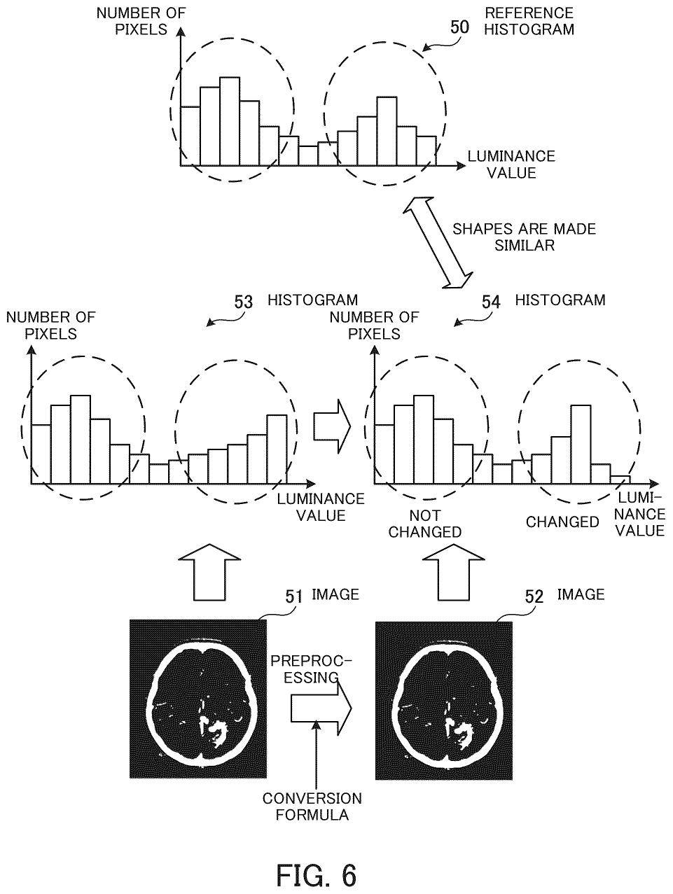

FIG. 6 is a diagram illustrating an example of preprocessing that improves the similarity between histograms of luminance values. For example, a reference histogram 50 is determined in advance. The reference histogram 50 is, for example, a histogram of an image from which good determination accuracy is expected. This is, for example, an image captured under an inspection condition under which the largest number of annotated images have been acquired.

The server 100 generates a conversion formula for making the shape of a histogram 53 of the luminance values of an image 51 similar to that of the reference histogram 50. The server 100 performs preprocessing for converting the luminance values of the image 51 in accordance with the conversion formula, and generates a preprocessed image 52.

In this preprocessing, for example, the server 100 does not change the luminance values in a range of low luminance values but changes the luminance values in a range of high luminance values. Thus, the shape of a histogram 54 of the luminance values of the image 52 is made similar to the shape of the reference histogram 50.

By executing the same preprocessing for each of the images used for learning and the images used for inference, the histograms of the luminance values of all the images are made similar to the reference histogram 50. As a result, the histograms of the luminance values of all the images become similar. By using images having similar histograms in the learning phase of machine learning, it is possible to generate an accurate learned model. Then, when image diagnosis is performed by using the learned model, preprocessing of converting a diagnosis target image with a conversion formula corresponding to the imaging conditions used when the image has been captured is performed. In this way, it is possible to accurately perform image diagnosis on the image.

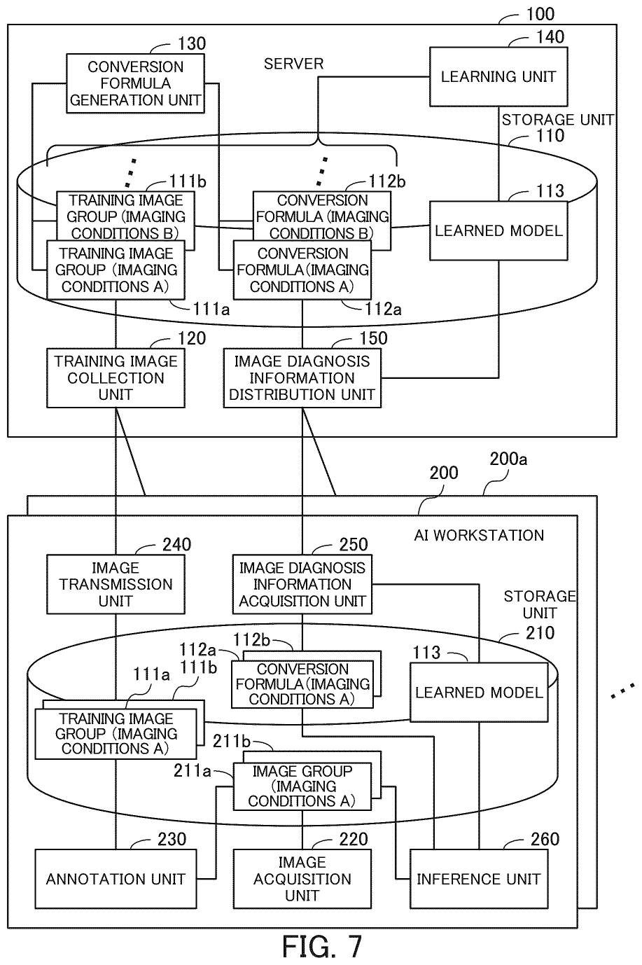

FIG. 7 is a block diagram illustrating functions of an individual apparatus for image diagnosis support. The server 100 includes a storage unit 110, a training image collection unit 120, a conversion formula generation unit 130, a learning unit 140, and an image diagnosis information distribution unit 150.

The storage unit 110 stores information used for machine learning. For example, the storage unit 110 stores training image groups 111a, 111b, etc., acquired from the AI workstations 200, 200a, etc., installed in the plurality of facilities 30, 31, etc., respectively. The training image groups 111a, 111b, etc., are images to which training data is added by annotation. Imaging conditions are set for images included in their respective training image groups 111a, 111b, etc. The imaging conditions include the type of the imaging apparatus, the dose, the type of the applied filtering process, and the like. The storage unit 110 also stores conversion formulas 112a, 112b, etc., for their respective imaging conditions. The conversion formulas 112a, 112b, etc., are formulas for calculating post-conversion luminance values from pre-conversion luminance values in order to improve the similarity among the histograms of the luminance values of the images. Further, the storage unit 110 stores a learned model 113. The learned model 113 is, for example, a neural network model generated based on the training image groups 111a, 111b, etc., and the conversion formulas 112a, 112b, etc.

The training image collection unit 120 acquires training image groups 111a, 111b, etc., from the AI workstations 200, 200a, etc., of the facilities 30, 31, etc., respectively. The training image collection unit 120 stores the acquired training image groups 111a, 111b, etc., in the storage unit 110.

The conversion formula generation unit 130 generates the conversion formulas 112a, 112b, etc., for the training image groups 111a, 111b, etc., respectively. For example, the conversion formula generation unit 130 determines the reference histogram 50 and generates the conversion formulas 112a, 112b, etc., that make the histograms of the luminance values of the training image groups 111a, 111b, etc., similar to the reference histogram 50.

The learning unit 140 executes the learning phase of machine learning based on the training image groups 111a, 111b, etc., and the conversion formulas 112a, 112b, etc., and generates the learned model 113. For example, the learning unit 140 generates the learned model 113 that determines whether or not a lesion appears in an image and, if the lesion appears, what disease the lesion represents. The learning unit 140 stores the generated learned model 113 in the storage unit 110.

The image diagnosis information distribution unit 150 distributes image diagnosis information to the AI workstations 200, 200a, etc., of the facilities 30, 31, etc., respectively. For example, the image diagnosis information distribution 150 transmits, to each of the AI workstations 200, 200a, etc., the conversion formula used in the corresponding AI workstation and the learned model 113.

The AI workstation 200 includes a storage unit 210, an image acquisition unit 220, an annotation unit 230, an image transmission unit 240, an image diagnosis information acquisition unit 250, and an inference unit 260.

The storage unit 210 stores unannotated image groups 211a and 211b and annotated training image groups 111a and 111b, in addition to the conversion formulas 112a and 112b and the learned model 113 generated by the server 100.

The image group 211a is a plurality of images captured by the examination apparatus 30a. The image group 211b is a plurality of images captured by the examination apparatus 30b. It is assumed that the examination apparatus 30a and the examination apparatus 30b have different imaging conditions. The image groups 211a and 211b are provided with, for example, information indicating their respective imaging conditions.

The training image group 111a is obtained by adding annotation results to the images extracted from the image group 211a. The training image group 111b is obtained by adding annotation results to the images extracted from the image group 211b.

The image acquisition unit 220 acquires images from the examination apparatuses 30a and 30b. For example, the image acquisition unit 220 acquires the captured images and the imaging conditions from the examination apparatus 30a, and stores the images to which the imaging conditions are added in the storage unit 210 as the image group 211a. The image acquisition unit 220 acquires the captured images and the imaging conditions from the examination apparatus 30b, and stores the images to which the imaging conditions are added in the storage unit 210 as the image group 211b.

The annotation unit 230 supports annotation for at least a part of the image groups 211a and 211b. For example, the annotation unit 230 displays an image selected by a doctor on the monitor 30c. The doctor interprets the displayed image and makes a diagnosis such as the presence or absence of a lesion. When the doctor inputs the diagnosis result, the annotation unit 230 stores the image to which the diagnosis result is added in the storage unit 210. For example, when the annotation is performed on an image extracted from the image group 211a, the annotation unit 230 stores the image to which the diagnosis result is added in the storage unit 210 as an image in the training image group 111a. When the annotation is performed on an image extracted from the image group 211b, the annotation unit 230 stores the image to which the diagnosis result is added in the storage unit 210 as an image in the training image group 111b.

The image transmission unit 240 transmits the training image groups 111a and 111b to the server 100, for example, in response to an image transmission instruction from the user. The image transmission unit 240 may periodically transmit untransmitted training images in the training image groups 111a and 111b to the server 100.

The image diagnosis information acquisition unit 250 acquires the conversion formulas 112a and 112b and the learned model 113 from the server 100 as image diagnosis information. The image diagnosis information acquisition unit 250 stores the acquired conversion formulas 112a and 112b and learned model 113 in the storage unit 210.

The inference unit 260 executes the AI inference phase based on the conversion formulas 112a and 112b and the learned model 113, and estimates the presence or absence of a lesion appearing in the images in the image groups 211a and 211b, the detected lesion type, and the like. The inference unit 260 displays, for example, the estimation result on the monitor 30c.

Note that the lines connecting the elements illustrated in FIG. 7 indicate part of the communication paths, and communication paths other than the illustrated communication paths may also be set. The function of an element illustrated in FIG. 7 may be implemented by, for example, causing a computer to execute a program module corresponding to the element.

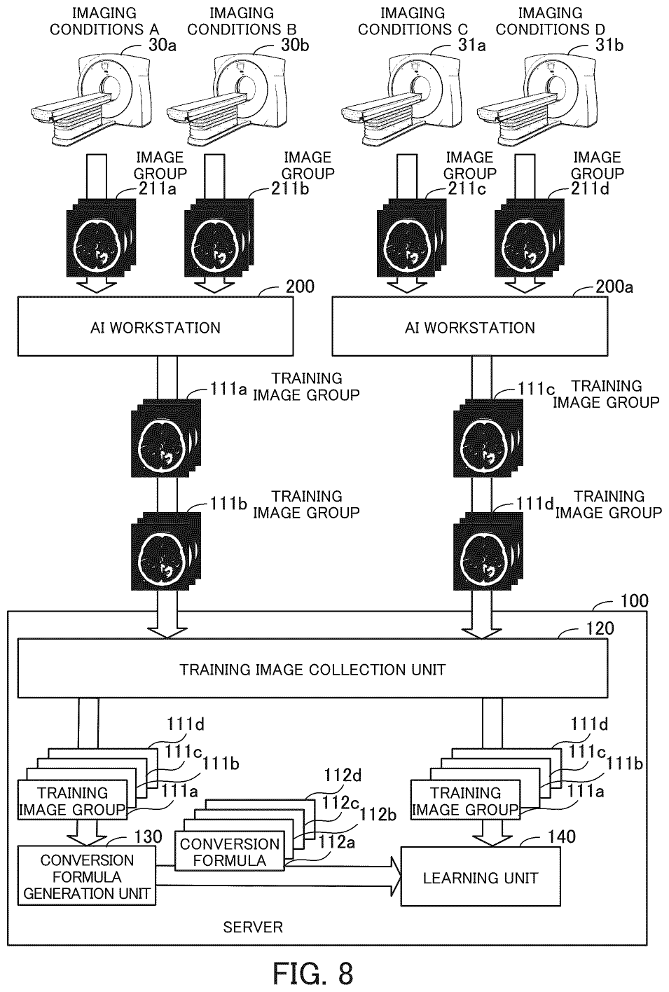

FIG. 8 is a diagram illustrating an example of an image diagnosis support method using images captured under different imaging conditions. In the example of FIG. 8, four examination apparatuses 30a, 30b, 31a, and 31b, that is, the examination apparatuses 30a and 30b installed in the facility 30 and the examination apparatuses 31a and 31b installed in the facility 31, perform imaging under their respective imaging conditions. For example, the examination apparatus 30a transmits the image group 211a captured under imaging conditions A to the AI workstation 200. The examination apparatus 30b transmits the image group 211b captured under imaging conditions B to the AI workstation 200. The examination apparatus 31a transmits an image group 211c captured under imaging conditions C to the AI workstation 200a. The examination apparatus 31b transmits an image group 211d captured under imaging conditions D to the AI workstation 200a.

The AI workstation 200 generates the training image groups 111a and 111b based on images extracted from the image groups 211a and 211b, respectively. The AI workstation 200 transmits the generated training image groups 111a and 111b to the server 100. Similarly, the AI workstation 200a generates training image groups 111c and 111d based on images extracted from the image groups 211c and 211d, respectively. The AI workstation 200a transmits the generated training image groups 111c and 111d to the server 100.

In the server 100, the training image collection unit 120 acquires the training image groups 111a to 111d. The training image groups 111a to 111d acquired by the training image collection unit 120 are passed to the conversion formula generation unit 130 and the learning unit 140 via the storage unit 110.

The conversion formula generation unit 130 generates conversion formulas 112a to 112d for their respective training image groups 111a to 111d. The conversion formulas 112a to 112d generated by the conversion formula generation unit 130 are passed to the learning unit 140 via the storage unit 110.

The conversion formulas 112a to 112d for their respective training image groups 111a to 111d are conversion formulas for converting the luminance values of the respective images so as to increase the similarity among the histograms of the luminance values of the images in the training image groups 111a to 111d. For example, the conversion formula generation unit 130 determines a histogram of luminance values of a certain image as the reference histogram 50. Then, the conversion formula generation unit 130 generates the conversion formulas 112a to 112d so as to maximize the similarity among the converted histograms of the images included in the training image groups 111a to 111d and the reference histogram 50.

When the histogram of an image included in one of the training image groups 111a to 111d is determined as the reference histogram, there is no need to generate a conversion formula corresponding to this training image group.

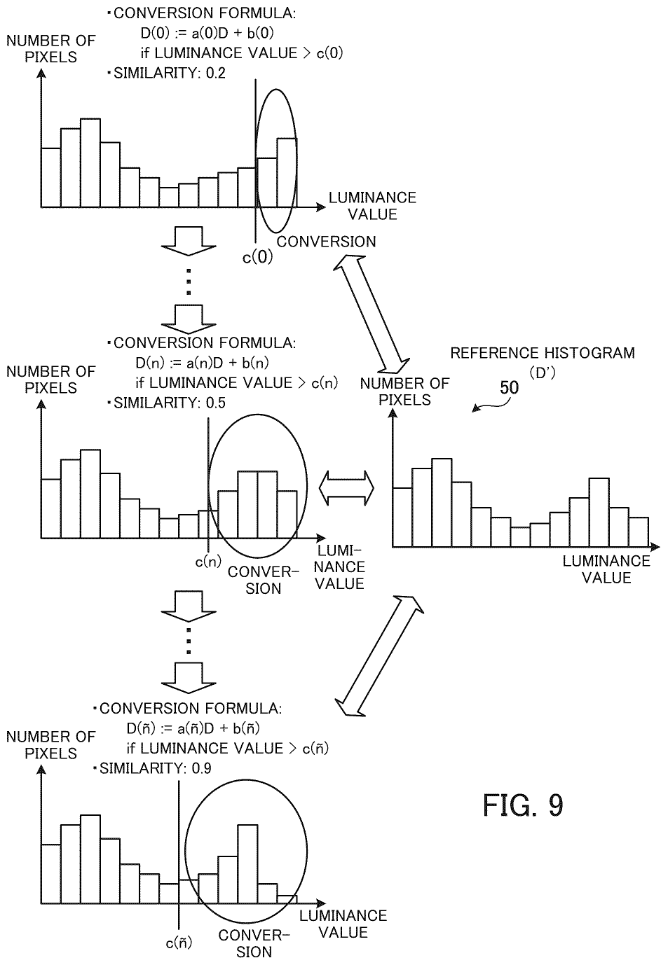

FIG. 9 is a diagram illustrating an example of a procedure for generating a conversion formula. In the example in FIG. 9, the distribution of the pixel values of the reference histogram 50 is D′. Also, let D be the distribution of pixel values of the average histogram before conversion of each image of the training image group for which a conversion formula is to be generated. Here, the conversion formula generation unit 130 generates a linear function as a conversion formula for converting the distribution D into D′. When the post-conversion distribution in the n-th optimization step (n is an integer of 0 or more) is D(n), the conversion formula may be expressed as follows, for example.

D(n)=a(n)D+b(n) if luminance value d>c(n) (1)

D(n)=D if luminance value d≤c(n) (2)

Equation (1) represents that, for each pixel of the distribution D in which the luminance value d is higher than c(n), the post-conversion luminance value d′ of the luminance value d of the pixel is expressed by “d′=a(n) d+b(n)”. Equation (2) indicates that the luminance value is not converted for a pixel whose luminance value d is equal to or less than c(n). That is, the range in which the luminance value is larger than c(n) is the range of the luminance values to be converted. Each of a(n), b(n), and c(n) is a constant (real number) calculated in the optimization process in the n-th optimization step.

In the optimization process, a(n), b(n), and c(n) are optimized so that the converted distribution has the maximum similarity to the distribution D′ of the reference histogram 50. The similarity between two distributions of luminance values may be calculated by a method such as Jensen Shannon divergence or Kullback Leibler divergence.

In the example of FIG. 9, initial values a(0) and b(0) of a(n) and b(n) are set to “a(0)=1” and “b(0)=0”. In this case, D (0)=D. The similarity between D and D′ is “0.2”.

The conversion formula generation unit 130 solves an optimization problem for obtaining the values of a(n), b(n), and c(n) that maximize the similarity. For example, in the first optimization step, the conversion formula generation unit 130 obtains a(1), b(1), and c(1) obtained by correcting the initial values a(0), b(0), and c(0) so as to increase the similarity, according to a predetermined optimization method. Then, the conversion formula generation unit 130 calculates the similarity between the post-conversion distribution D (1) and the distribution D′ of the reference histogram 50 by using a(1), b(1), and c(1). The conversion formula generation unit 130 repeats the optimization step as described above until it is determined that the similarity does not increase any more. As the optimization method, a hill-climbing method, a gradient method, or the like may be used.

In the example of FIG. 9, the similarity between the distribution D (n) obtained by conversion with the conversion formula calculated in the n-th optimization step and the distribution D′ of the reference histogram 50 is “0.5”. The optimization step is further repeated, and the similarity between the distribution D (n) (n is with a tilde) obtained by conversion with the conversion formula calculated in the n-th (with a tilde) optimization step and the distribution D′ of the reference histogram 50 reaches “0.9”.

By optimizing the three constants in this manner, it is possible to obtain a conversion formula that maximizes the similarity of the distribution with respect to the reference histogram 50. The conversion formula generation unit 130 performs such conversion formula generation processing for each of the training image groups 111a to 111d. As a result, the conversion formulas 112a to 112d are obtained for their respective training image groups 111a to 111d.

Generation of a conversion formula is performed as the preprocessing phase before the AI-based learning phase and inference phase.

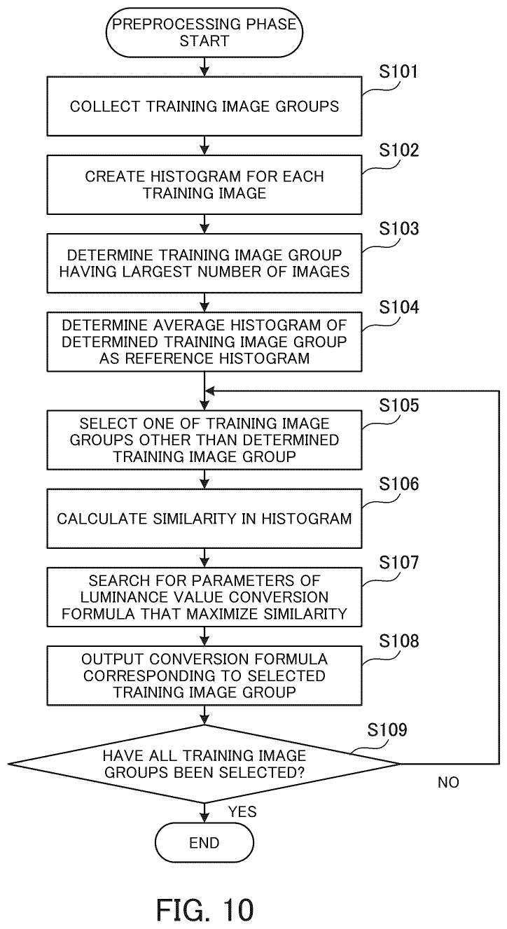

FIG. 10 is a flowchart illustrating an example of a processing procedure in the preprocessing phase. Hereinafter, the processing illustrated in FIG. 10 will be described in order of step numbers.

[Step S101] The training image collection unit 120 collects training image groups from a plurality of AI workstations of the plurality of facilities 30, 31, etc.

[Step S102] The conversion formula generation unit 130 creates a histogram of luminance values for each of the training images included in the plurality of training image groups. For example, the conversion formula generation unit 130 counts the number of pixels for each luminance value, and arranges the numbers in ascending order according to the luminance value. Note that the conversion formula generation unit 130 may divide the entire range of possible luminance values into a predetermined number of ranges and may count the number of pixels for each luminance value included in these divided ranges.

[Step S103] The conversion formula generation unit 130 determines a training image group having the largest number of images.

[Step S104] The conversion formula generation unit 130 determines an average histogram of the determined training image group as a reference histogram. The number of pixels for each luminance value of the average histogram is obtained, for example, by summing the number of pixels for each luminance value of a plurality of images and dividing the total value by the number of images.

[Step S105] The conversion formula generation unit 130 selects one of the training image groups other than the training image group determined in step S103.

[Step S106] The conversion formula generation calculates the similarity between the average unit 130 histogram of the selected training image group and the reference histogram.

[Step S107] The conversion formula generation unit 130 searches for the parameters of the luminance value conversion formula that maximizes the similarity. For example, the conversion formula generation unit 130 repeats a process of optimizing the parameters “a(n) and b(n)” of the linear function and the parameter “c(n)” indicating the threshold value for the luminance values to be converted until a predetermined termination condition is satisfied. When the termination condition is satisfied, a set of parameters having the highest similarity in histogram among the sets of parameters “a(n), b(n), and c(n)” generated so far are set as the optimum values of the parameters.

For example, the termination condition is satisfied when the difference between the similarity of the histogram of the image converted by applying the parameters obtained in the latest optimization step and the similarity of the histogram in the previous optimization step is equal to or less than a predetermined value. Alternatively, the termination condition may be determined to be satisfied when the number of iterations of the optimization step has reached a predetermined number.

[Step S108] The conversion formula generation unit 130 outputs a conversion formula corresponding to the selected training image group. For example, the conversion formula generation unit 130 stores the optimized conversion formula in the storage unit 110 in association with the imaging conditions of the selected training image group. The conversion formula generation unit 130 may store only the values of the optimized parameters as the conversion formula.

[Step S109] The conversion formula generation unit 130 determines whether or not all the training image groups other than the training image group determined in step S103 have been selected. When all the training image groups have been selected, the conversion formula generation unit 130 ends the preprocessing phase. If any training image group remains unselected, the conversion formula generation unit 130 advances the process to step S105.

In this way, in the preprocessing phase, a conversion formula is generated for each training image group captured under the same imaging conditions. By using the generated conversion formula, it is possible to make the histograms of luminance values of a plurality of images captured under different imaging conditions similar to each other. After the preprocessing phase is completed, the learning phase is performed.

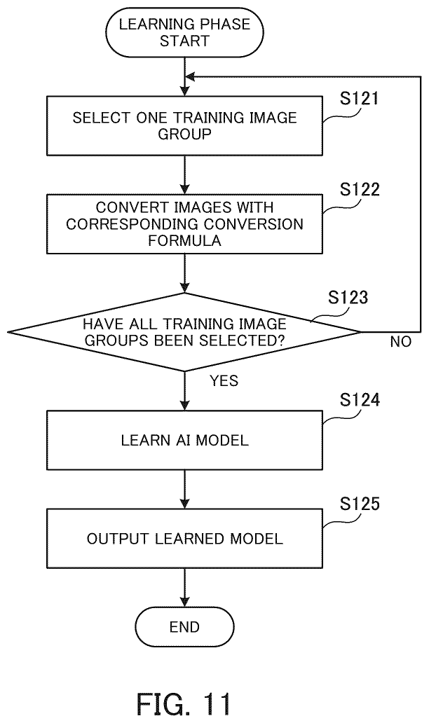

FIG. 11 is a flowchart illustrating an example of a processing procedure of the learning phase. Hereinafter, the processing illustrated in FIG. 11 will be described in order of step numbers.

[Step S121] The learning unit 140 selects one training image group.

[Step S122] The learning unit 140 converts the luminance values of all images included in the selected training image group by using a conversion formula corresponding to the training image group. When the selected training image group is the training image group from which the reference histogram has been generated, the learning unit 140 advances the processing to the next step without performing the luminance value conversion processing.

[Step S123] The learning unit 140 determines whether or not all the training image groups have been selected. If all the training image groups have been selected, the learning unit 140 advances the process to step S124. If any training image group remains unselected, the learning unit 140 advances the process to step S121.

[Step S124] The learning unit 140 performs learning of an AI model, using all the images included in the training image groups obtained after the luminance value conversion process. For example, the learning unit 140 performs learning of a neural network model. In this case, the learning unit 140 optimizes the weight parameters of the neural network such that, when an individual image is input, the same result as the annotation result assigned to the individual image is obtained.

[Step S125] The learning unit 140 outputs the learned model 113 generated by the learning. For example, the learning unit 140 stores the learned model 113 in the storage unit 110.

In this way, the learned model 113 is generated by using a large number of images having similar histograms of luminance values. As a result, an accurate learned model is generated.

When the learning phase is completed, the image diagnosis information distribution unit 150 distributes the search expression and the learned model as the image diagnosis information to the AI workstations of the plurality of facilities 30, 31, etc.

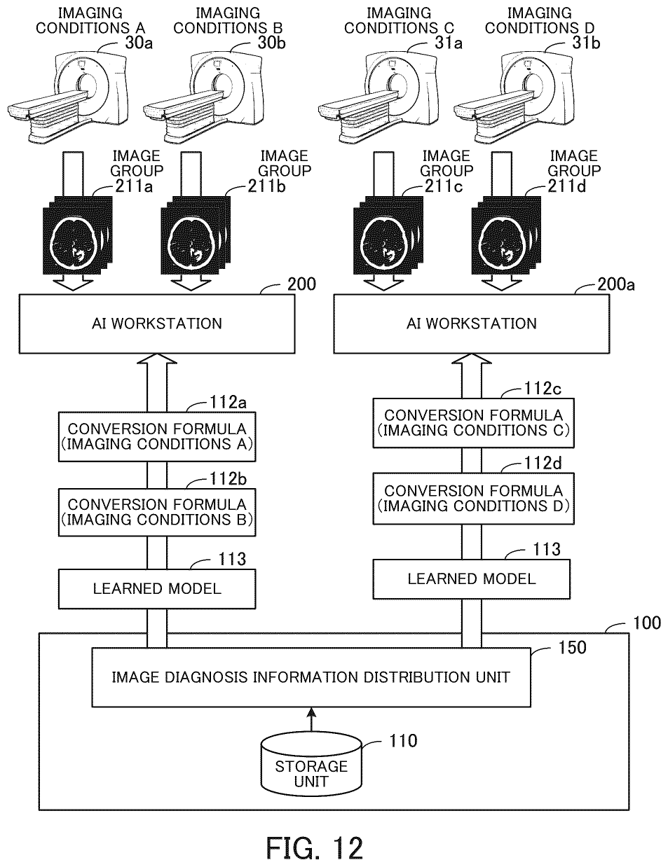

FIG. 12 is a diagram illustrating an example of image diagnosis information distribution processing. The AI workstation 200 has acquired the image group 211a captured under the imaging conditions A from the examination apparatus 30a, and has acquired the image group 211b captured under the imaging conditions B from the examination apparatus 30b. So, the image diagnosis information distribution unit 150 transmits the conversion formula 112a corresponding to the imaging conditions A, the conversion formula 112b corresponding to the imaging conditions B, and the learned model 113 to the AI workstation 200.

The AI workstation 200a has acquired the image group 211c captured under the imaging conditions C from the examination apparatus 31a, and has acquired the image group conditions D from the 211d captured under the imaging examination apparatus 31b. So, the image diagnosis information distribution unit 150 transmits the conversion formula 112c corresponding to the imaging conditions C, the conversion formula 112d corresponding to the imaging conditions D, and the learned model 113 to the AI workstation 200a.

Each of the AI workstations 200 and 200a performs inference on an unannotated image by using the acquired image diagnosis information.

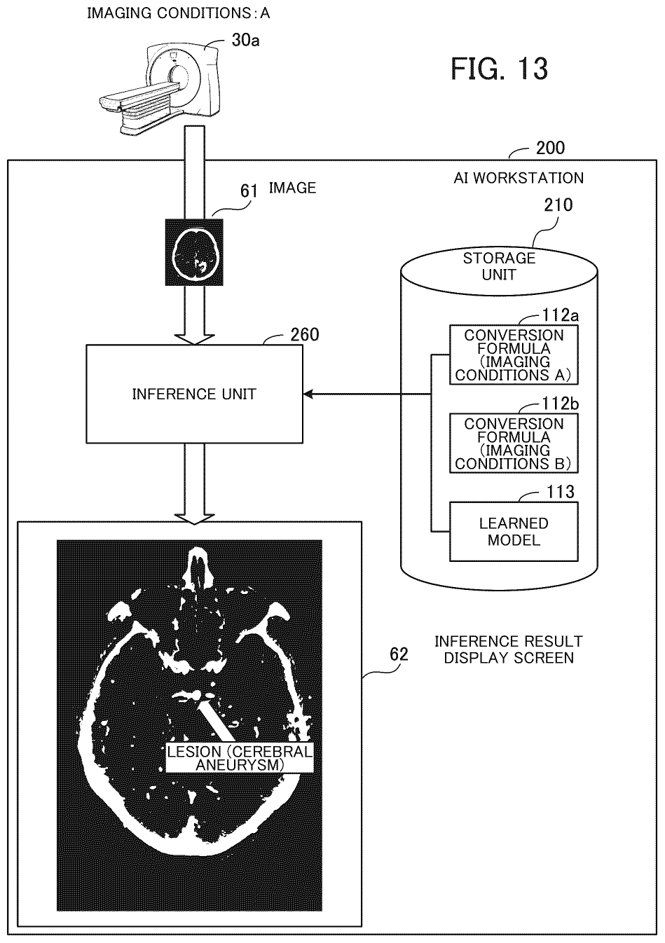

FIG. 13 is a diagram illustrating an example of inference processing. For example, it is assumed that the AI workstation 200 performs inference on an image 61 acquired from the examination apparatus 30a. In this case, the inference unit 260 of the AI workstation 200 infers whether or not a lesion appears in the image 61, and if a lesion appears, infers the type of lesion, by using the conversion formula 112a corresponding to the imaging conditions A of the examination apparatus 30a and the learned model 113. Then, the inference unit 260 causes the monitor 30c to display an inference result display screen indicating the inference result. For example, when a lesion is found, the position of the lesion and the type of the lesion are displayed on the inference result display screen 62.

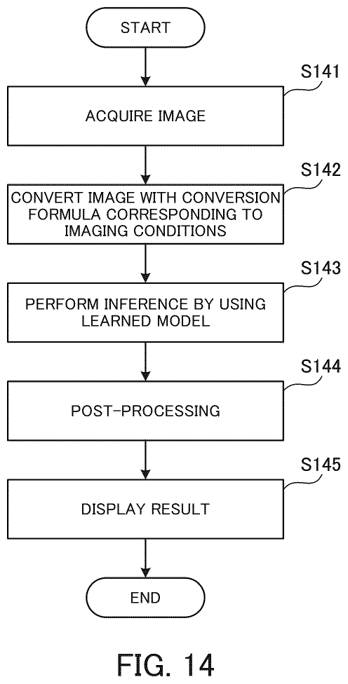

FIG. 14 is a flowchart illustrating an example of a processing procedure of the inference phase. Hereinafter, the processing illustrated in FIG. 14 will be described in order of step numbers.

[Step S141] The inference unit 260 acquires an image to be processed in the inference phase from the unannotated image groups 211a and 211b. For example, the inference unit 260 acquires an image specified by the user.

[Step S142] The inference unit 260 converts the luminance values of the image by using a conversion formula corresponding to the imaging conditions of the acquired image. For example, when the acquired image is an image that has been captured by the examination apparatus 30a, the inference unit 260 converts the image by using a conversion formula corresponding to the imaging conditions A.

[Step S143] The inference unit 260 performs an inference process on the image by using the learned model 113. For example, when the learned model 113 is a neural network, the inference unit 260 inputs an image to an input layer of the neural network and performs calculation according to the neural network. The inference unit 260 acquires an output value from an output layer of the neural network as the inference result.

[Step S144] The inference unit 260 performs post-processing of the inference processing. For example, the inference unit 260 performs processing such as threshold processing or Non-Maximum Suppression (NMS) as the post-processing.

[Step S145] The inference unit 260 displays the inference result on the monitor 30c.

In this way, by converting an inference target image with the conversion formula matching the imaging conditions under which the image has been captured and by performing inference, the histograms of the images input to the learned model 113 at the time of inference are unified. As a result, a decrease in the accuracy of inference due to a difference in imaging conditions is suppressed.

### OTHER EMBODIMENTS

In the second embodiment, an example of image diagnosis support using AI has been described. However, similar processing may also be applied to machine learning using a plurality of images generated under different conditions. For example, images captured for quality inspection of products in different manufacturing lines in a factory are different in appearance depending on the direction of the camera, ambient brightness, and the like. In this case, a conversion formula that maximizes the similarity among the histograms of the images is obtained, and the luminance values of the images are converted by the conversion formula in the learning/inference phase, thereby improving the detection accuracy in determining the quality of products.

Further, for example, by converting (inversely converting) images under certain imaging conditions from which a large amount of data has been obtained by using a conversion formula, it is also possible to artificially increase the number of images under imaging conditions, the number of images being a smaller amount of data than the images under the certain imaging conditions (data extension). In this case, the number of image data usable as the training data increases in the training of a machine learning model for the images under the imaging conditions for which only a small amount of data has been acquired. Accordingly, generalization performance and robustness in inference using the trained machine learning model are improved, and as a result, accuracy of inference using the machine learning model is improved.

In the second embodiment, conversion formulas are generated by using the annotated training image groups 111a, 111b, etc., but the results of the annotations are not used to generate the conversion formulas. Therefore, it is also possible to generate conversion formulas by using the image groups 211a, 211b, etc., on which no annotation has been performed.

In one mode, the difference in appearance among images used for machine learning is reduced.

All examples and conditional language provided herein are intended for the pedagogical purposes of aiding the reader in understanding the invention and the concepts contributed by the inventor to further the art, and are not to be construed as limitations to such specifically recited examples and conditions, nor does the organization of such examples in the specification relate to a showing of the superiority and inferiority of the invention. Although one or more embodiments of the present invention have been described in detail, it should be understood that various changes, substitutions, and alterations could be made hereto without departing from the spirit and scope of the invention.

What is claimed is:

1. A non-transitory computer-readable recording medium storing therein a computer program that causes a computer to execute a process comprising:
generating a histogram indicating a number of pixels for an individual luminance value of a first image obtained by capturing an image of a first subject under a first imaging condition;
generating a histogram indicating a number of pixels for an individual luminance value of a second image obtained by capturing an image of a second subject of a same type as the first subject under a second imaging condition;
generating a conversion rule for the luminance values of the pixels of the second image, the conversion rule improving a similarity between the histogram of the first image and the histogram of the second image;
converting a luminance value of an individual pixel of a third image obtained by capturing an image of a third subject of the same type as the first subject under the second imaging condition, by using the conversion rule; and
executing machine learning by using the third image whose luminance values have been converted.

2. The non-transitory computer-readable recording medium according to claim 1,
wherein the generating of the conversion rule includes generating a conversion rule in which a range of the luminance values to be converted is limited, and
wherein the converting of the luminance value of the individual pixel of the third image includes converting the luminance value of the individual pixel within the range limited in the conversion rule by using the conversion rule.

3. The non-transitory computer-readable recording medium according to claim 1, wherein the generating of the conversion rule includes optimizing a constant included in a conversion formula for the luminance values of the pixels of the second image by repeatedly executing a correction process that improves a similarity in shape between the histogram of the first image and the histogram of the second image, and includes using the conversion formula including the optimized constant as the conversion rule.

4. The non-transitory computer-readable recording medium according to claim 3,
wherein the generating of the conversion rule includes optimizing a first constant included in the conversion formula for calculating a post-conversion luminance value from a pre-conversion luminance value, and includes optimizing a second constant indicating a range of the luminance values to be converted, and
wherein the converting of the luminance value of the individual pixel of the third image includes converting luminance values of pixels in the range indicated by the second constant by using conversion formula.

5. The non-transitory computer-readable recording medium according to claim 1, wherein the generating of the histogram indicating the number of pixels for the individual luminance value of the first image includes referring to a number of images captured under respective imaging conditions, determining one of the plurality of imaging conditions as the first imaging condition based on the number of images, and determining an image captured under the first imaging condition as the first image.

6. The non-transitory computer-readable recording medium according to claim 1, wherein the executing of the machine learning with the third image whose luminance values have been converted includes training a machine learning model for detecting a certain part from an input image, by using the first image, the second image whose luminance values have been converted, or the third image whose luminance values have been converted.

7. The non-transitory computer-readable recording medium according to claim 6, wherein the process further comprises:
converting a luminance value of an individual pixel of a fourth image obtained by capturing an image of a subject of the same type as the first image under the second imaging condition by using the conversion rule; and
detecting the certain part from the fourth image whose luminance values have been converted, by using the trained machine learning model.

8. An image processing method comprising:
generating, by a processor, a histogram indicating a number of pixels for an individual luminance value of a first image obtained by capturing an image of a first subject under a first imaging condition;
generating, by the processor, a histogram indicating a number of pixels for an individual luminance value of a second image obtained by capturing an image of a second subject of a same type as the first subject under a second imaging condition;
generating, by the processor, a conversion rule for the luminance values of the pixels of the second image, the conversion rule improving a similarity between the histogram of the first image and the histogram of the second image;
converting, by the processor, a luminance value of an individual pixel of a third image obtained by capturing an image of a third subject of the same type as the first subject under the second imaging condition, by using the conversion rule; and
executing, by the processor, machine learning by using the third image whose luminance values have been converted.

9. An information processing apparatus comprising:
a memory; and
a processor coupled to the memory and the processor configured to:
generate a histogram indicating a number of pixels for an individual luminance value of a first image obtained by capturing an image of a first subject under a first imaging condition,
generate a histogram indicating a number of pixels for an individual luminance value of a second image obtained by capturing an image of a second subject of a same type as the first subject under a second imaging condition,
generate a conversion rule for the luminance values of the pixels of the second image, the conversion rule improving a similarity between the histogram of the first image and the histogram of the second image,
convert a luminance value of an individual pixel of a third image obtained by capturing an image of a third subject of the same type as the first subject under the second imaging condition, by using the conversion rule, and
execute machine learning by using the third image whose luminance values have been converted.
